(19) United States
(12) Patent Application Publication
(10) Pub. No.: US 2005/0272664 A1
(43) Pub. Date: Dec. 8, 2005

(54) Methods for inhibiting angiogenesis and or lymphangiogenesis

(75) Inventors: Bradley McColl, Victoria (AU); Steven Stacker, Victoria (AU); Marc Achen, Victoria (AU)

Correspondence Address: CROWELL & MORING LLP;INTELLECTUAL PROPERTY GROUP, P.O. BOX 14300, WASHINGTON, DC, 20044-4300, US

(73) Assignee: Ludwig Institute for Cancer Research, New York, NY (US)

(21) Appl. No.: 11/133,415

(22) Filed: May 20, 2005

Related U.S. Application Data
(60) Provisional application No. 60/572,469, filed on May 20, 2004.

(51) Int. Cl. A61K 38/06

(52) U.S. Cl. 514/18

(57) ABSTRACT

Proprotein convertase inhibitor has been found to block proteolytic processing and activation of VEGF-C and VEGF-D and inhibit angiogenesis and/or lymphangiogenesis. Method and composition are disclosed for inhibiting angiogenesis and/or lymphangiogenesis, and for treating conditions associated with excessive angiogenesis, such as tumors and/or retinopathies, as well as conditions associated with lymphangiogenesis, such as the metastatic spread of malignancies, macular degeneration, inflammatory mediated diseases, rheumatoid arthritis, diabetic retinopathy and psoriasis in a patient. The inventive method and composition utilize proprotein convertase antagonist selected from the group consisting of an anti-proprotein convertase antibody, an antisense nucleic acid molecule against a polynucleotide coding for a proprotein convertase, and an siRNA for inhibiting proprotein convertase expression, as well as proprotein convertase inhibitors.

### CROSS REFERENCE TO RELATED APPLICATIONS

This application claims the priority of U.S. Provisional Application No. 60/572,469, filed May 20, 2004, the disclosure of which is expressly incorporated by reference herein.

### BACKGROUND OF THE INVENTION

Vascular endothelial growth factor-C (VEGF-C) and VEGF-D are secreted glycoproteins that bind and activate VEGF receptor-2 (VEGFR-2) and VEGFR-3 (Achen et al., Proc. Natl. Acad. Sci. USA 95: 548-553, 1998; Joukov et al., EMBO J. 15: 290-298, 1996), cell surface receptor tyrosine kinases expressed predominantly on blood vascular and lymphatic endothelia respectively (for review see Stacker et al., FASEB J. 16: 922-934, 2002). VEGFR-3 signals for lymphangiogenesis (growth of lymphatic vessels) (Veikkola et al., EMBO J. 20: 1223-1231, 2001) whereas VEGFR-2 is thought to signal for angiogenesis (growth of blood vessels). Human VEGF-C and VEGF-D stimulate both angiogenesis and lymphangiogenesis in vivo (Byzova et al., Blood 99: 4434-4442, 2002; Veikkola et al., EMBO J. 20: 1223-1231, 2001; Marconcini et al., Proc. Natl. Acad. Sci. USA 96: 9671-9676, 1999; Rissanen et al., Circ. Res. 92: 1098-1106, 2003; Bhardwaj et al., Human Gene Therapy 14: 1451-1462, 2003; Rutanen et al., Circulation 109: 1029-1035, 2004; Cao et al., Proc. Natl. Acad. Sci. USA 95: 14389-14394, 1998; Jeltsch et al., Science 276: 1423-1425, 1997).

Importantly, the angiogenesis induced by VEGF-C and VEGF-D in tumors can promote solid tumor growth and metastatic spread, and the lymphangiogenesis induced by these growth factors promotes metastatic spread of tumor cells to the lymphatic vessels and lymph nodes (Skobe et al., Nature Med. 7: 192-198, 2001; Stacker et al., Nature Med. 7: 186-191, 2001; Mandriota et al., EMBO J. 20: 672-682, 2001; Karpanen et al., Cancer Res. 61: 1786-1790, 2001; Skobe et al., Am. J. Pathol. 159: 893-903, 2001). Furthermore, clinicopathological data indicate a role for these growth factors in a range of prevalent human cancers. For example, VEGF-D expression was reported to be an independent prognostic factor for both overall and disease-free survival in colorectal cancer (White et al., Cancer Res. 62: 1669-1675, 2002) and levels of VEGF-C mRNA in lung cancer are associated with lymph node metastasis (Niki et al., Clin. Cancer Res. 6: 2431-2439, 2000) and in breast cancer correlate with lymphatic vessel invasion and shorter disease-free survival (Kinoshita et al., Breast Cancer Res. Treat. 66: 159-164, 2001; for review see Stacker et al., FASEB J. 16: 922-934, 2002 and Stacker et al., Nature Rev. Cancer 2: 573-583, 2002).

VEGF-C and VEGF-D are each secreted from the cell in a full-length form containing an N-terminal propeptide, a C-terminal propeptide and a central VEGF homology domain (VHD) containing the binding sites for VEGFR-2 and VEGFR-3 (Joukov et al., EMBO J. 16: 3898-3911, 1997; Stacker et al., J. Biol. Chem. 274: 32127-32136, 1999). Subsequently, the propeptides are proteolytically cleaved from the VHD to generate a mature form, consisting of dimers of the VHD, that binds VEGFR-2 and VEGFR-3 with high affinity. The affinities of the mature form of VEGF-D for VEGFR-2 and VEGFR-3 are approximately 290-fold and 40-fold greater, respectively, than those of the unprocessed form (Stacker et al., J. Biol. Chem. 274: 32127-32136, 1999) and similar increases in receptor affinity due to processing were observed for VEGF-C (Joukov et al., EMBO J. 16: 3898-3911, 1997). Thus, the proteolytic processing of VEGF-C and VEGF-D is a mechanism for activating these growth factors.

The proprotein convertases are a family of proteases which process precursor proteins by cleaving after the consensus sequence Arg-Xaa-(Lys/Arg)-Arg (for review see Nakayama, Biochem J. 327: 625-635, 1997). This consensus sequence is similar to the sites at which VEGF-C and VEGF-D are cleaved. Furin, the first member of the proprotein convertases to be identified, is potently inhibited by the substrate analogue Decanoyl-Arg-Val-Lys-Arg-chloromethylketone (Dec-RVKR-CMK) (Steineke-Grober et al., EMBO J. 11: 2407-2414, 1992; Sugrue et al., J. Gen. Virol. 82: 1375-1386, 2001; Hallenberger et al., Nature 360: 358-361 1992).

### SUMMARY OF THE INVENTION

It has now been found that treatment with a proprotein convertase inhibitor can block proteolytic processing and activation of VEGF-C and VEGF-D and inhibit angiogenesis and/or lymphangiogenesis.

By reason of their inhibition of angiogenesis and/or lymphangiogenesis, proprotein convertase inhibitors are useful to treat conditions associated with excessive angiogenesis, such as tumors and/or retinopathies, as well as conditions associated with lymphangiogenesis, such as the metastatic spread of malignancies, macular degeneration, inflammatory mediated diseases, rheumatoid arthritis, diabetic retinopathy and psoriasis in a patient.

In one embodiment, this invention provides a method of inhibiting angiogenesis or lymphangiogenesis comprising administering to an organism in need thereof an effective angiogenesis or lymphangiogenesis inhibiting amount of a proprotein convertase antagonist selected from the group consisting of an anti-proprotein convertase antibody, an antisense nucleic acid molecule against a polynucleotide coding for a proprotein convertase, and an siRNA for inhibiting proprotein convertase expression.

Also provided are pharmaceutical compositions comprising an effective angiogenesis or lymphangiogenesis inhibiting amount of a proprotein convertase inhibitor, or an antagonist selected from the group consisting of an anti-proprotein convertase antibody, an antisense nucleic acid molecule against a polynucleotide coding for a proprotein convertase, and an siRNA for inhibiting proprotein convertase expression, and a pharmaceutically acceptable excipient.

### BRIEF DESCRIPTION OF THE DRAWINGS

The invention will be described in further detail hereinafter with reference to representative embodiments illustrated by the accompanying drawing figures in which:

FIG. 1 is a Western blot analysis of human VEGF-C secreted from cells expressing a tagged, full-length VEGF-C following treatment of the cells with a representative proprotein convertase inhibitor; and

FIG. 2 is a Western blot analysis of human VEGF-D secreted from cells expressing a tagged, full-length VEGF-D following treatment of the cells with a representative proprotein convertase inhibitor.

FIG. 3 shows a schematic map of VEGF-DΔC and VEGF-DΔN (upper panel) and Western blots of the immunoprecipitated VEGF-D proteins from the 293EBNA VEGF-DΔC cells (middle panel) and from the 293EBNA VEGF-DΔN cells (lower panel).

FIG. 4 shows Western blots comparing the VEGF-D species immunoprecipitated from the conditioned media of (A) 293EBNA and (B) LoVo cells after transient transfection with VEGF-D expression constructs.

FIG. 5 shows Western blot analysis of conditioned media of LoVo cells co-transfected with VEGF-D expression constructs with PC expression constructs.

### DETAILED DESCRIPTION OF THE INVENTION

A number of proprotein convertase inhibitors are known. Examples of such inhibitors include inhibitory prosegments of proprotein convertases, inhibitory variants of anti-trypsin and peptidyl haloalkylketone inhibitors. Representative inhibitory prosegments of proprotein convertases include the inhibitory prosegments of PC5A (also known as PC6A), PC5B (also known as PC6B), PACE4, PC1 (also known as PC3), PC2, PC4, PC7 and Furin (Thomas, Nature Reviews Mol. Cell Biol. 3 (2002) 753-766; Zhong et al., J. Biol. Chem. 274: 33913-33920, 1999). A representative inhibitory variant of anti-trypsin is α-1 antitrypsin Portland, an engineered variant of naturally occurring antitrypsin that inhbits multiple proprotein convertases (Jean et al., Proc. Natl Acad. Sci. USA 95 (1998) 7293-7298). Representative peptidyl halomethyl ketone inhibitors include decanoyl-Arg-Val-Lys-Arg-chloromethylketone (Dec-RVKR-CMK), decanoyl-Phe-Ala-Lys-Arg-chloromethylketone (Dec-FAKR-CMK), decanoyl-Arg-Glu-Ile-Arg-chloromethylketone (Dec-REIR-CMK), and decanoyl-Arg-Glu-Lys-Arg-chloromethylketone (Dec-REKR-CMK) (Stieneke-Grober, A. et al., EMBO J. 11 (1992) 2407-2414; Jean et al., Proc. Natl Acad. Sci. USA 95 (1998) 7293-7298; Garten W. et al., Virology 72 (1989) 25-31). For purposes of illustration, the invention will be described hereinafter by reference to exemplary tests using the representative substrate analogue Dec-RVKR-CMK without limiting the invention thereto.

These inhibitors of proprotein convertases, such as Dec-RVKR-CMK or the inhibitory prosegments of PCs, can be used to block the activation of VEGF-C and VEGF-D and thereby inhibit angiogenesis, lymphangiogenesis and other biological effects induced by partially processed or fully processed VEGF-C or VEGF-D. A particularly relevant clinical setting for use of the invention is in the treatment of cancer in which angiogenesis and lymphangiogenesis induced by these growth factors can promote solid tumor growth and/or metastatic spread. However, the invention is also useful in other situations in which angiogenesis and/or lymphangiogenesis underlies the pathology, e.g macular degeneration in the eye. Other applications include treatment of inflammatory mediated diseases, rheumatoid arthritis, diabetic retinopathy and psoriasis.

Because only the fully processed forms of VEGF-C and VEGF-D bind to VEGF Receptor 2 (VEGFR-2), as well as exhibiting increased binding to VEGF Receptor 3 (VEGFR-3), it is possible by use of proprotein convertase inhibitors to alter the processing of the full length growth factors and thereby modulate the relative rates of VEGFR-2 and VEGFR-3 activation. In this way, one can affect the balance between angiogenesis and lymphangiogenesis through administration of proprotein convertase inhibitors.

The proprotein convertase inhibitors may be administered by known routes of local or systemic administration such as injection to the desired site of action or intravenous administration. The inhibitors may be administered in admixture with known carriers and or adjuvants, as well as with other active agents for the treatment of pathological conditions associated with angiogenesis and/or lymphangiogenesis. Dosage regimens may be determined by persons skilled in the art. For treatment of a human patient, a typical dosage will lie between 0.1 μg and 100 mg per kilogram of body weight.

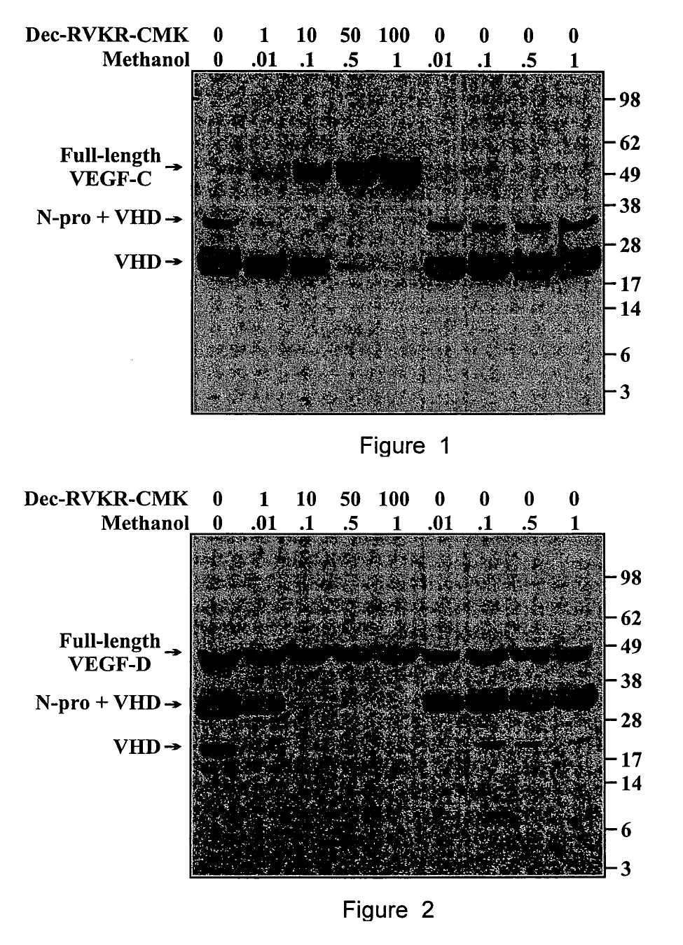

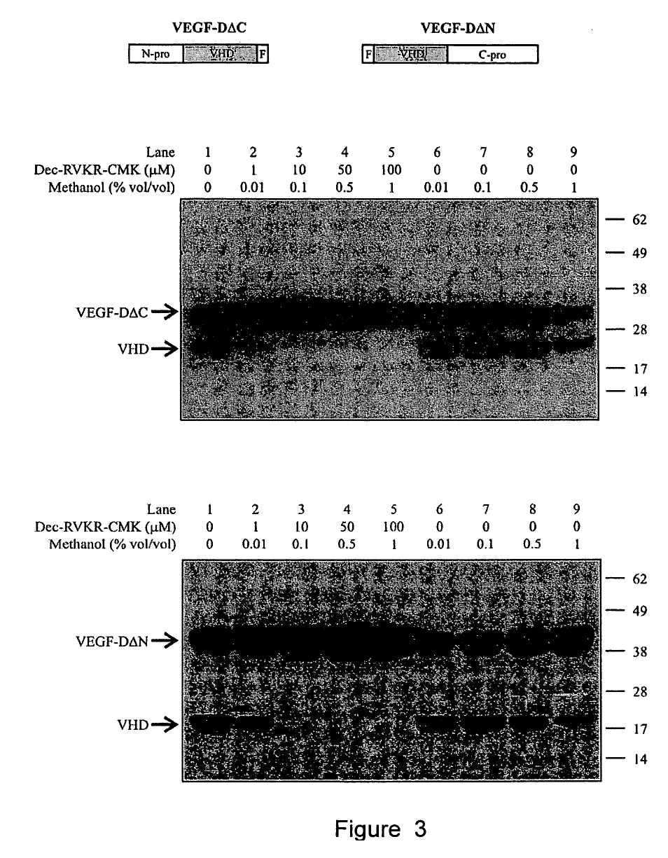

FIGS. 1 and 2 depict the Western blot analysis of human VEGF-C (FIG. 1) and VEGF-D (FIG. 2) secreted from 293EBNA cells expressing VEGF-C-FULL-N-Myc or VEGF-D-FULL-N-FLAG respectively, following treatment with Dec-RVKR-CMK. The numbers at the top of each figure denote the concentration of Dec-RVKR-CMK (μM), and the concentration of methanol (% v/v). Identities of the various VEGF-C and VEGF-D species are shown to the left. Numbers to the right denote positions of molecular weight markers (kDa).

In addition to the inhibitors described above or otherwise known to those skilled in the art, neutralizing antibodies may also be used to inhibit the biological action (e.g. the function or expression) of proprotein convertase (“the target protein”). In one embodiment of the invention, antisense oligonucleotides are used as antagonizing agents. The antisense oligonucleotides preferably inhibit target expression by inhibiting translation of the target. In a further embodiment, the antagonizing agent is small interfering RNAs (siRNA, also known as RNAi, RNA interference nucleic acids). siRNA are double-stranded RNA molecules, typically 21 n.t. in length, that are homologous to a gene or polynucleotide that encodes the proprotein convertase (“the target gene”) and interfere with the target gene's expression. Also provided are methods of inhibiting the target gene expression or target protein function utilizing DNA enzymes; ribozymes and triplex-forming nucleic acid molecules, as will be described in more details below.

An antibody suitable for the present invention may be a polyclonal antibody. Preferably, the antibody is a monoclonal antibody. The antibody may also be isoform-specific. The monoclonal antibody or binding fragment thereof of the invention may be Fab fragments, F(ab)2 fragments, Fab′ fragments, F(ab′)2 fragments, Fd fragments, Fd′ fragments or Fv fragments. Domain antibodies (dAbs) (for review, see Holt et al., 2003, Trends in Biotechnology 21:484-490) are also suitable for the methods of the present invention.

Various methods of producing antibodies with a known antigen are well-known to those ordinarily skilled in the art (see for example, Harlow and Lane, 1988, Antibodies: A Laboratory Manual. Cold Spring Harbor Laboratory Press, Cold Spring Harbor, N.Y.; see also WO 01/25437). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In accordance with the present invention, the antibodies or binding fragments thereof may be characterized as those which are capable of specific binding to a target protein or an antigenic fragment thereof, preferably an epitope that is recognized by an antibody when the antibody is administered in vivo. Antibodies can be elicited in an animal host by immunization with a target protein-derived immunogenic component, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

The antibodies may be from humans, or from animals other than humans, preferably mammals, such as rat, mouse, guinea pig, rabbit, goat, sheep, and pig. Preferred are mouse monoclonal antibodies and antigen-binding fragments or portions thereof. In addition, chimeric antibodies and hybrid antibodies are embraced by the present invention. Techniques for the production of chimeric antibodies are described in e.g. Morrison et al., 1984, Proc. Natl. Acad. Sci. USA, 81:6851-6855; Neuberger et al., 1984, Nature, 312:604-608; and Takeda et al., 1985, Nature, 314:452-454. For human therapeutic purposes, humanized, or more preferably, human antibodies are used.

Further, single chain antibodies are also suitable for the present invention (e.g., U.S. Pat. Nos. 5,476,786 and 5,132,405 to Huston; Huston et al., 1988, Proc. Natl. Acad. Sci. USA, 85:5879-5883; U.S. Pat. No. 4,946,778 to Ladner et al.; Bird, 1988, Science, 242:423-426 and Ward et al., 1989, Nature, 334:544-546). Single chain antibodies are formed by linking the heavy and light immunoglobulin chain fragments of the Fv region via an amino acid bridge, resulting in a single chain polypeptide. Univalent antibodies are also embraced by the present invention.

Many routes of delivery are known to the skilled artisan for delivery of anti-target antibodies. For example, direct injection may be suitable for delivering the antibody to the site of interest. It is also possible to utilize liposomes with antibodies in their membranes to specifically deliver the liposome to the area where target gene expression or function is to be inhibited. These liposomes can be produced such that they contain, in addition to monoclonal antibody, other therapeutic agents, such as those described above, which would then be released at the tumor site (e.g., Wolff et al., 1984, Biochem. et Biophys. Acta, 802:259).

This invention also provides antisense nucleic acid molecules and compositions comprising such antisense molecules. The constitutive expression of antisense RNA in cells has been known to inhibit the gene expression, possibly via the blockage of translation or prevention of splicing. Interference with splicing allows the use of intron sequences which should be less conserved and therefore result in greater specificity, inhibiting expression of a gene product of one species but not its homologue in another species.

The term antisense component corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vivo conditions. This antisense molecule must have sufficient complementarity, about 18-30 nucleotides in length, to the target gene so that the antisense RNA can hybridize to the target gene (or mRNA) and inhibit target gene expression regardless of whether the action is at the level of splicing, transcription, or translation. The antisense components of the present invention may be hybridizable to any of several portions of the target cDNA, including the coding sequence, 3′ or 5′ untranslated regions, or other intronic sequences, or to target mRNA.

Antisense RNA is delivered to a cell by transformation or transfection via a vector, including retroviral vectors and plasmids, into which has been placed DNA encoding the antisense RNA with the appropriate regulatory sequences including a promoter to result in expression of the antisense RNA in a host cell. In one embodiment, stable transfection and constitutive expression of vectors containing target cDNA fragments in the antisense orientation are achieved, or such expression may be under the control of tissue or development-specific promoters. Delivery can also be achieved by liposomes.

For in vivo therapy, the currently preferred method is direct delivery of antisense oligonucleotides, instead of stable transfection of an antisense cDNA fragment constructed into an expression vector. Antisense oligonucleotides having a size of 15-30 bases in length and with sequences hybridizable to any of several portions of the target cDNA, including the coding sequence, 3′ or 5′ untranslated regions, or other intronic sequences, or to target mRNA, are preferred. Sequences for the antisense oligonucleotides to target are preferably selected as being the ones that have the most potent antisense effects. Factors that govern a target site for the antisense oligonucleotide sequence include the length of the oligonucleotide, binding affinity, and accessibility of the target sequence. Sequences may be screened in vitro for potency of their antisense activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides.

The preferred target antisense oligonucleotides are those oligonucleotides which are stable, have a high resistance to nucleases, possess suitable pharmacokinetics to allow them to traffic to target tissue site at non-toxic doses, and have the ability to cross through plasma membranes.

Phosphorothioate antisense oligonucleotides may be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

The delivery route will be the one that provides the best antisense effect as measured according to the criteria described above. In vitro and in vivo assays using antisense oligonucleotides have shown that delivery mediated by cationic liposomes, by retroviral vectors and direct delivery are efficient. Another possible delivery mode is targeting using antibody to cell surface markers for the target cells. Antibody to target or to its receptor may serve this purpose.

Alternatively, nucleic acid sequences which inhibit or interfere with gene expression (e.g., siRNA, ribozymes, aptamers) can be used to inhibit or interfere with the activity of RNA or DNA encoding a target protein.

siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48; Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411:428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerheadribozyme compositions of the invention (see below).

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:863-74). The effectiveness of the siRNA may be assessed by any of a number of assays following introduction of the dsRNAs. These include Western blot analysis using antibodies which recognize the target gene product following sufficient time for turnover of the endogenous pool after new protein synthesis is repressed, reverse transcriptase polymerase chain reaction and Northern blot analysis to determine the level of existing target mRNA.

Further compositions, methods and applications of siRNA technology are provided in U.S. Pat. Nos. 6,278,039, 5,723,750 and 5,244,805, which are incorporated herein by reference.

Ribozymes are enzymatic RNA molecules capable of catalyzing specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach, 1988, Nature 334:585-591; and PCT Application. No. WO89/05852, the contents of which are incorporated herein by reference. Hammerhead ribozyme sequences can be embedded in a stable RNA such as a transfer RNA (tRNA) to increase cleavage efficiency in vivo (Perriman et al., 1995, Proc. Natl. Acad. Sci. USA, 92:6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al., 1998, Nature 393:284-9; Kuwabara et al., 1998, Nature Biotechnol. 16:961-5; and Kuwabara et al., 1998, Mol. Cell 2:617-27; Koseki et al., 1999, J. Virol 73:1868-77; Kuwabara et al., 1999, Proc. Natl. Acad. Sci. USA, 96:1886-91; Tanabe et al., 2000, Nature 406:473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

The ribozymes of the present invention also include RNA endoribonucleases (“Cech-type ribozymes”) such as the one which occurs naturally in Tetrahymena thermophila (known as the IVS, or L-19 IVS RNA) and which has been extensively described in Zaug, et al., 1984, Science, 224:574-578; Zaug, et al., 1986, Science 231:470-475; Zaug, et al., 1986, Nature 324:429-433; published International patent application No. WO88/04300; and Been, et al., 1986, Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or siRNA are substantially the same sequence of at least 5 and preferably 6, 7, 8, 9, 10, 11, 12, 13, 14, 15, 16, 17, 18, 19 or 20 or more contiguous nucleotides of a target nucleic acid.

In a long target RNA chain, significant numbers of target sites are not accessible to the ribozyme because they are hidden within secondary or tertiary structures (Birikh et al., 1997, Eur. J. Biochem. 245:1-16). To overcome the problem of target RNA accessibility, computer generated predictions of secondary structure are typically used to identify targets that are most likely to be single-stranded or have an “open” configuration (see Jaeger et al., 1989, Methods Enzymol. 183:281-306). Other approaches utilize a systematic approach to predicting secondary structure which involves assessing a huge number of candidate hybridizing oligonucleotides molecules (see Milner et al., 1997, Nat. Biotechnol. 15: 537-41; and Patzel and Sczakiel, 1998, Nat. Biotechnol. 16:64-8). Additionally, U.S. Pat. No. 6,251,588, the contents of which are herein incorporated by reference, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the siRNA oligonucleotides and ribozymes of the invention.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C., 1991, Anticancer Drug Des., 6:569-84; Helene, C., et al., 1992, Ann. N.Y. Acad. Sci., 660:27-36; and Maher, L. J., 1992, Bioassays 14:807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of target gene. DNA enzymes incorporate some of the mechanistic features of both antisense and ribozyme technologies. DNA enzymes are designed so that they recognize a particular target nucleic acid sequence, much like an antisense oligonucleotide. They are, however, catalytic and specifically cleave the target nucleic acid.

There are currently two basic types of DNA enzymes, both of which were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo are similar methods of delivery RNA ribozyme, as outlined in detail above. Additionally, one of skill in the art will recognize that, like antisense oligonucleotide, DNA enzymes can be optionally modified to improve stability and improve resistance to degradation.

The dosage ranges for the administration of the antagonists of the invention are those large enough to produce the desired effect. The dosage should not be so large as to cause adverse side effects, such as unwanted cross-reactions, anaphylactic reactions, and the like. Generally, the dosage will vary with the age, condition, sex and extent of disease of the patient and can be determined by one of skill in the art. The dosage can be adjusted by the individual physician in the event of any complication.

The antagonists of the invention can be administered parenterally by injection or by gradual perfusion over time. The antagonists can be administered intravenously, intraperitoneally, intramuscularly, subcutaneously, intracavity, or transdermally.

Another embodiment of the present invention relates to pharmaceutical compositions comprising one or more proprotein convertase inhibitor, or an antibody against a proprotein convertase, a suitable antisense nucleic acid molecule against a polynucleotide coding for a proprotein convertase, and an siRNA for inhibiting proprotein convertase expression, together with a physiologically- and/or pharmaceutically-acceptable carrier, excipient, or diluent. Physiologically acceptable carriers, excipients, or stabilizers are known to those skilled in the art (see Remington's Pharmaceutical Sciences, 17th edition, (Ed.) A. Osol, Mack Publishing Company, Easton, Pa., 1985). Acceptable carriers, excipients or stabilizers are nontoxic to recipients at the dosages and concentrations-employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

### EXAMPLES

### Example 1

Inhibition of Proteolytic Processing of VEGF-C and VEGF-D by Dec-RVKR-CMK

293EBNA cells stably transfected with an expression construct encoding VEGF-C-FULL-N-Myc (full-length VEGF-C tagged with Myc at the N-terminus) or VEGF-D-FULL-N-FLAG (full-length VEGF-D tagged with FLAG at the N-terminus) secrete fully and partially processed forms of VEGF-C or VEGF-D into the culture medium (Jukov et al., EMBO J. 16:3898-3911 1997; Stacker et al., J. Biol. Chem. 274: 32127-32136, 1999). These cells were grown overnight in 24-well plates, seeded with 8×10⁴ cells per well. At the time when cultures were initiated in these plates, growth medium was supplemented with Dec-RVKR-CMK (Calbiochem) (dissolved in methanol) to a final concentration of 0, 1, 10, 50 or 100 μM. Solvent control cultures were also established, in which the medium was supplemented with methanol at concentrations equal to those present in the cultures treated with inhibitor. After 18 hours of growth (at 37° C., 10% CO₂ in a humidified environment), conditioned cell culture medium was removed from the cells and cleared by centrifugation (5 minutes, 250×g, 4° C.).

VEGF-C species were immunoprecipitated from conditioned medium by addition of anti-VEGF-C antibody that binds the VHD and C-terminal propeptide (R&D Systems). VEGF-D species were immunoprecipitated by addition of A2 antiserum that binds within the VHD of VEGF-D (Stacker et al., J. Biol. Chem. 274: 32127-32136, 1999). Immune complexes were allowed to form for 2 hours at 4° C. with gentle agitation and precipitated by addition of 10 μl of protein A sepharose beads and incubation for 2 hours at 4° C. with gentle agitation. Protein A sepharose complexes were recovered by centrifugation (5 minutes, 250×g, 4° C.) and washed twice with ice cold 50 mM Tris, 150 mM NaCl, pH 8.0. The precipitated VEGF-C and VEGF-D species were then analysed by Western blot using biotinylated antibodies against the VHD and C terminus of VEGF-C, or the VHD of VEGF-D (R&D Systems) and streptavidin-horse radish peroxidase conjugate (Zymed) (FIGS. 1 and 2).

FIG. 1 shows a Western blot of immunoprecipitated samples from 293 VEGF-C-FULL-N-Myc cells. The bands in the control sample (extreme left lane) have been characterised previously (Joukov et al., EMBO J. 16: 3898-3911 1997), and are as follows: the 50 kDa band is full-length VEGF-C, the 33 kDa band is the VHD bound to the N-terminal propeptide (N-pro) and the 22 kDa band is the mature VEGF-C subunit consisting of the VHD.

FIG. 2 shows a Western blot of immunoprecipitated samples from 293 VEGF-D-FULL-N-FLAG cells. The bands in the control sample (extreme left lane) have been characterised previously (Stacker et al., J. Biol. Chem. 274: 32127-32136, 1999) and are as follows: the 48 kDa band is full-length VEGF-D, the 33 kDa species is the VHD bound to the N-terminal propeptide (N-pro) and the 21 kDa band is the mature VEGF-D subunit consisting of the VHD.

As a result of treatment of 293EBNA cells expressing either VEGF-C-FULL-N-Myc or VEGF-D-FULL-N-FLAG with Dec-RVKR-CMK, the fully and partially processed forms of VEGF-C and VEGF-D are dramatically reduced in abundance in a dose-dependent manner. At the highest concentration of Dec-RVKR-CMK (100 μM), only the full-length form of VEGF-D was detected indicating that proteolytic processing had been totally blocked. Cells treated with methanol did not show altered processing of VEGF-C or VEGF-D.

These results demonstrate that treatment with a proprotein convertase inhibitor can block proteolytic processing and activation of VEGF-C and VEGF-D. Thus, by blocking this activation in vivo with proprotein convertase inhibitors, it is possible to treat conditions associated with VEGF-C and/or VEGF-D activity, for example, conditions associated with angiogenesis and/or lymphangiogenesis.

### Example 2

An Inhibitor of All PCs Blocks Processing of VEGF-D at Both the N-Terminal and C-Terminal Sites of Cleavage

VEGF-D is proteolytically processed at the N- and C-termini of the VHD to generate a mature form consisting of dimers of the VHD (Stacker et al, J. Biol. Chem. 274 32127-32136, 1999). The effect of Dec-RVKR-CMK, an inhibitor of all PCs (Jean et al, Proc. Natl. Acad. Sci. USA 95 7293-7298 1998), on the processing of VEGF-D derivatives by 293EBNA cells was monitored to establish if PCs can carry out both of these cleavage events.

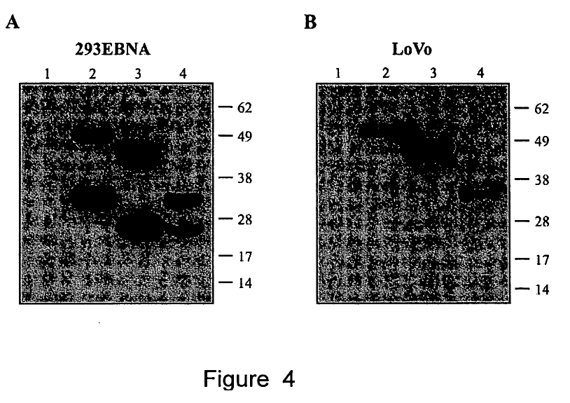

An expression plasmid encoding a truncated derivative of human VEGF-D consisting of the N-terminal propeptide and the VHD tagged at the carboxyl terminus with the FLAG epitope (referred to as VEGF-DΔC) (FIG. 3, top panel) has been previously described (Stacker et al, J. Biol. Chem. 274 32127-32136, 1999). The VEGF-DΔC protein construct facilitates study of cleavage of the N-terminal propeptide from the VHD. The conditioned media of 293EBNA cells stably transfected with the VEGF-DΔC expression plasmid contain a mixture of VEGF-D proteins, including unprocessed VEGF-DΔC, and the proteolytically processed VHD. A second cell line was generated by stable transfection of 293EBNA cells with an expression construct encoding VEGF-DΔN, a truncated derivative of human VEGF-D consisting of the VHD tagged at the amino terminus with the FLAG epitope and C-terminal propeptide (FIG. 3, top panel). This protein construct facilitates study of cleavage of the C-terminal propeptide from the VHD. The conditioned media of 293EBNA cells expressing VEGF-DΔN contain a mixture of VEGF-D proteins, consisting of unprocessed VEGF-DΔN, and the proteolytically processed VHD. The VEGF-DΔN expression plasmid was constructed by replacing the 2-kb EcoRV fragment of the pAPEX-3 construct for full-length VEGF-D with the 2-kb EcoRV fragment of the pAPEX-3 construct for VEGF-DΔNΔC-FLAG (Stacker et al, J. Biol. Chem. 274 32127-32136, 1999).

The 293EBNA cell lines expressing VEGF-DΔC or VEGF-DΔN were seeded into 24-well plates (8×10⁴ cells/well), in medium supplemented with Dec-RVKR-CMK dissolved in methanol, to a final concentration of 0, 1, 10, 50 or 100 μM. After 18 hours incubation (37° C., 10% CO₂ in an humidified incubator), conditioned cell culture media were removed and clarified by centrifugation (5 minutes, 250×g, 4° C.). VEGF-D species were immunoprecipitated from conditioned media of 293EBNA VEGF-DΔC cells by addition of M2-agarose beads (Sigma-Aldrich), that bind the FLAG epitope attached to the carboxyl-terminus of the VHD, and from conditioned media of 293EBNA VEGF-DΔN cells by addition of A2 antiserum, that binds to epitopes within the VHD (Stacker et al, J. Biol. Chem. 274 32127-32136, 1999), followed by incubation with protein-A sepharose beads. Beads and bound proteins were recovered by centrifugation (5 minutes, 250×g, 4° C.) and washed twice with Tris-buffered saline (TBS; 50 mM Tris-Cl, 150 mM NaCl, pH 8.0). The precipitated VEGF-D species were then analysed by reducing SDS-PAGE and Western blotting using a biotinylated antibody against the VHD of human VEGF-D (R&D Systems), and Streptavidin-horse radish peroxidase conjugate (Zymed).

FIG. 3 shows a schematic map of VEGF-DΔC and VEGF-DΔN (upper panel) and Western blots of the immunoprecipitated VEGF-D proteins from the 293EBNA VEGF-DΔC cells (middle panel) and from the 293EBNA VEGF-DΔN cells (lower panel). In the top panel, “F” denotes the FLAG peptide, “N-pro” the N-terminal propeptide and “C-pro” the C-terminal propeptide. For the middle panel, bands in the control sample (lane 1) consist of unprocessed VEGF-DΔC (˜33 kDa) and the VHD (˜21 kDa) generated by proteolytic removal of the N-terminal propeptide. Following treatment of the 293EBNA VEGF-DΔC cells with Dec-RVKR-CMK, the band representing mature VHD is reduced in abundance in a dose dependent manner (lanes 2-5). At the highest concentration of Dec-RVKR-CMK used (100 μM; lane 5) only unprocessed VEGF-DΔC is detected, indicating that proteolytic processing is completely blocked. For the lower panel, the bands in the control sample (lane 1) consist of unprocessed VEGF-DΔN (˜44 kDa) and the VHD (˜21 kDa) generated by proteolytic removal of the amino-terminal propeptide. Following treatment of the 293EBNA VEGF-DΔN cells with Dec-RVKR-CMK the band representing mature VHD is reduced in abundance in a dose dependent manner (lanes 2-5). At the highest concentration of Dec-RVKR-CMK used (100 μM; lane 5) only unprocessed VEGF-DΔN is detected, indicating that proteolytic processing is completely blocked. For both the middle and lower panels, cells treated with methanol alone, to control for solvent-specific effects, did not show altered processing of VEGF-D (lanes 6-9). Concentrations of Dec-RVKR-CMK (μM) and methanol (% vol/vol) are shown above these panels, the identities of VEGF-D species to the left and sizes of molecular weight standards (kDa) to the right.

These results show that an inhibitor of all the PCs blocks the cleavage of both the N- and C-terminal propeptides from the VHD of VEGF-D, indicating that members of this protease family may be important for activation of this growth factor.

### Example 3

VEGF-D is Not Processed in LoVo Cells Lacking Active Furin

The LoVo cell line is a human colon carcinoma line that lacks enzymatically active furin (Takahashi et al, Biochem. Biophys. Res. Commun. 195 1019-1026 1993; Takahashi et al, J. Biol. Chem. 270 26565-26569, 1995), a broadly expressed member of the proprotein convertases (Nakayama, Biochem. J. 327 625-635, 1997). As a result, LoVo cells fail to process numerous proproteins (Nakayama, Biochem. J. 327 625-635, 1997). Therefore, LoVo cells were analysed for the capacity to process VEGF-D to establish if this cell line could be used as a processing-deficient background in which to monitor the effect of individual PCs on the proteolytic activation of VEGF-D.

LoVo and 293EBNA cells were transiently transfected with expression constructs encoding full-length VEGF-D tagged at the amino terminus with the FLAG epitope (pVDAPEX FULL-N-FLAG), VEGF-DΔN tagged at the amino terminus with the FLAG epitope (pVDAPEXΔN), or VEGF-DΔC tagged at the carboxyl terminus with the FLAG epitope (pVDAPEXΔC) (Stacker et al, J. Biol. Chem. 274 32127-32136, 1999). After 48 hours incubation (37° C., 10% CO₂ in an humidified incubator), conditioned media were immunoprecipitated by addition of M2-agarose beads (Sigma-Aldrich) that bind the FLAG epitope. Immune complexes were allowed to form for two hours at 4° C. with gentle agitation. Beads and bound proteins were recovered by centrifugation (5 minutes, 250×g, 4° C.) and washed twice with Tris-buffered saline (TBS; 50 mM Tris-Cl, 150 mM NaCl, pH 8.0). The precipitated VEGF-D species were then analysed by reducing. SDS-PAGE and Western blotting using an M2-horse radish peroxidase conjugate (Sigma-Aldrich).

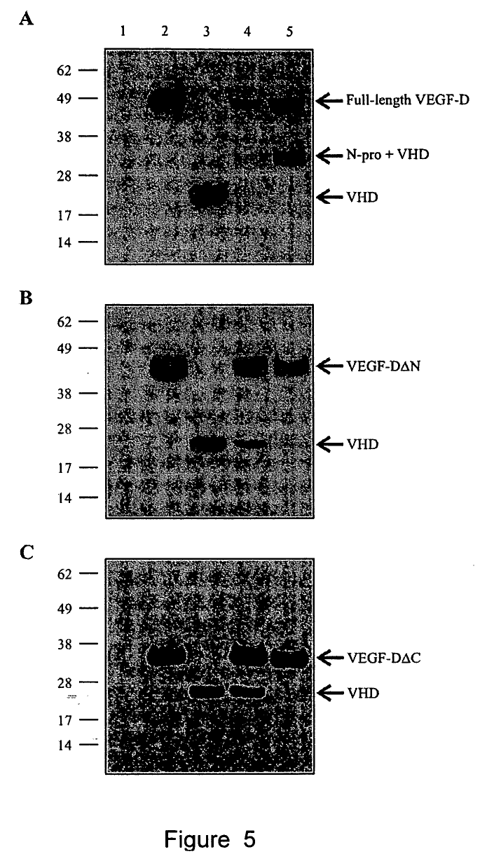

FIG. 4 shows Western blots comparing the VEGF-D species immunoprecipitated from the conditioned media of (A) 293EBNA and (B) LoVo cells after transient transfection with VEGF-D expression constructs. Expression constructs used to transfect cells are as follows: Lane 1 pAPEX3; lane 2 pVDAPEX FULL-N-FLAG; lane 3 pVDAPEXΔN; lane 4 pVDAPEXΔC. Proteolytic processing in 293EBNA cells leads to detection of unprocessed (˜48 kDa band in lane 2, ˜44 kDa band in lane 3 and ˜33 kDa band in lane 4) and processed VEGF-D derivatives (˜33 kDa band in lane2, ˜21 kDa band in lane 3 and ˜21 kDa band in lane 4). In contrast, when expressed in LoVo cells, only the unprocessed VEGF-D derivatives are detected. No processed forms of the proteins were detected in the conditioned media of the LoVo cells even after prolonged exposure of the blots. Sizes of molecular weight standards (kDa) are shown to the right.

These findings demonstrate that LoVo cells are incapable of processing VEGF-D and that this cell line will be an appropriate background for co-expression studies aimed at identifying the PCs capable of processing VEGF-D.

### Example 4

Processing of VEGF-D by Individual PCs

In order to establish which members of the PC family of proteases are capable of activating VEGF-D, LoVo cells were co-transfected with expression constructs for VEGF-D derivatives and for various PCs, and the transfected cells analysed for the capacity to process VEGF-D.

Proprotein convertase expression plasmids were constructed by polymerase chain reaction (PCR) amplification of the open reading frames of furin, PC5 and PC7. Cloned cDNAs encoding these proteases were supplied by the American Type Culture Collection and used as template DNA for the amplification reactions. Oligonucleotides used for amplification of open reading frames contained restriction enzyme sites to facilitate cloning of PCR products. PCR products were cloned into pcDNA3 (Invitrogen, Carlsbad USA) by digestion of PCR products with the appropriate restriction enzymes and ligation to similarly digested pcDNA3 with T4 DNA ligase. The amplified open reading frames of furin and PC7 were ligated into pcDNA3 after digestion with HindIII and XbaI. The amplified open reading frame of PC5 was ligated into pcDNA3 after digestion with NotI and XbaI. Recombinant plasmids shown to contain the desired DNA fragment by restriction mapping were sequenced on both strands to exclude the presence of mutations. The proprotein convertase expression constructs were designated pcDNA3:furin, pcDNA3:PC5 and pcDNA3:PC7.

The proprotein convertase expression constructs were used to co-transfect LoVo cells in combination with pVDAPEX FULL-N-FLAG, pVDAPEXΔN or pVDAPEXΔC. After 48 hours incubation (37° C., 10% CO₂ in an humidified incubator), conditioned media were removed and clarified by centrifugation (5 minutes, 250×g, 4° C.). VEGF-D species from cells transfected with PC expression constructs in combination with pVDAPEX FULL-N-FLAG were immunoprecipitated from conditioned media by addition of A2 antiserum that binds to epitopes within the VHD (Stacker et al, J. Biol. Chem. 274 32127-32136, 1999). Immune complexes were allowed to form for two hours at 4° C. with gentle agitation, and precipitated by addition of 10 μl of protein-A sepharose beads, and incubation for a further two hours as before. Conditioned media from LoVo cells transfected with PC expression constructs in combination with pVDAPEXΔN or pVDAPEXΔC were immunoprecipitated with M2-agarose beads (Sigma-Aldrich). Precipitated proteins were recovered by centrifugation (5 minutes, 250×g, 4° C.), washed twice with Tris-buffered saline (TBS; 50 mM Tris-Cl, 150 mM NaCl, pH 8.0) and analysed by reducing SDS-PAGE and Western blotting using a biotinylated antibody against the VHD of human VEGF-D (R&D Systems) followed by Streptavidin-horse radish peroxidase conjugate (Zymed) in the case of material from cells transfected with pVDAPEX FULL-N-FLAG, or using an M2-horse radish peroxidase conjugate (Sigma-Aldrich) in the case of material from cells transfected with pVDAPEXΔN or pVDAPEXΔC.

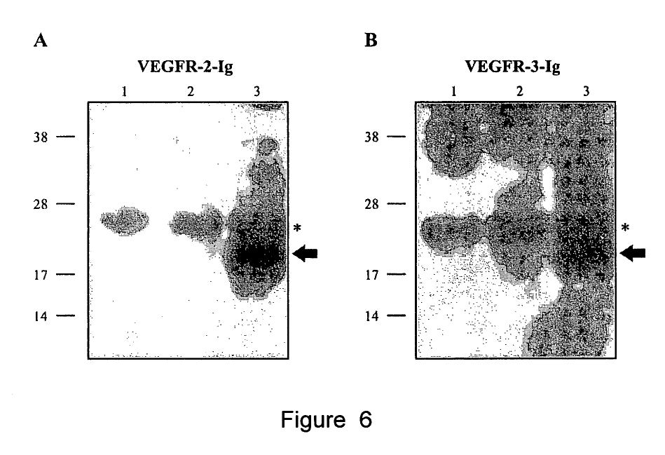

FIG. 5 shows Western blot analysis of conditioned media of LoVo cells co-transfected with VEGF-D expression constructs with PC expression constructs. Sizes of molecular weight standards (kDa) are shown to the left of the figures and the identities of the bands to the right. FIG. 5A shows the results of co-transfection of LoVo cells with pVDAPEX FULL-N-FLAG in combination with expression constructs encoding human furin, PC5 or PC7. The DNA combinations used to transfect LoVo cells were as follows: Lane 1, pAPEX3+pcDNA3; lane 2, pVDAPEX FULL-N-FLAG+pcDNA3; lane 3, pVDAPEX FULL-N-FLAG+pcDNA3:furin; lane 4, pVDAPEX FULL-N-FLAG+pcDNA3:PC5; lane 5, pVDAPEX FULL-N-FLAG+pcDNA3:PC7. No processing of VEGF-D-FULL-N-FLAG was observed in the absence of PC expression constructs (lane 2). The introduction of pcDNA3:furin resulted in almost complete conversion of the ˜48 kDa VEGF-D FULL-N-FLAG protein to the ˜21 kDa mature VHD species (lane 3). Co-transfection with pcDNA3:PC5 resulted in three fragments being detected in the conditioned medium, unprocessed VEGF-D FULL-N-FLAG (˜48 kDa), a partially processed form consisting of the amino-terminal propeptide and the VHD (˜33 kDa), and fully processed mature VHD (˜21 kDa) (lane 4). These findings indicate that furin and PC5 can cleave both propeptides for the VHD of VEGF-D. Co-transfection of pVDAPEX FULL-N-FLAG with pcDNA3:PC7 generated unprocessed protein and the partially processed ˜33 kDa form but not the mature VHD (lane 5) (the mature VHD was not detected even after prolonged exposure of the blot) suggesting that PC7 can remove the C-terminal propeptide, but not the N-terminal propeptide from the VHD.

FIG. 5B shows the results of co-transfection of LoVo cells with pVDAPEXΔN and expression constructs encoding furin, PC5 or PC7. DNA combinations used to transfect cells were as follows: Lane 1, pAPEX3+pcDNA3; lane 2, pVDAPEXΔN+pcDNA3; lane 3, pVDAPEXΔN+pcDNA3:furin; lane 4, pVDAPEXΔN+pcDNA3:PC5; lane 5, pVDAPEXΔN+pcDNA3:PC7. LoVo cells did not process VEGF-DΔN in the absence of PC expression constructs, as a single ˜44 kDa species was detected in the conditioned media (lane 2). Co-transfection of LoVo cells with pVDAPEXΔN and pcDNA3:furin resulted in complete conversion of VEGF-DΔN to the 21 kDa mature VHD species (lane 3). The presence of pcDNA3:PC5 or pcDNA3:PC7 resulted in a portion of VEGF-DΔN being processed to the mature VHD (lanes 4 and 5). These results demonstrate that furin, PC5 and PC7 are all capable of catalysing the proteolytic cleavage of the C-terminal propeptide from the VHD of VEGF-DΔN.

FIG. 5 C shows co-transfection of LoVo cells with pVDAPEXΔC and expression constructs encoding furin, PC5 or PC7. The DNA combinations used to transfect LoVo cells were as follows: Lane 1, pAPEX3+pcDNA3; lane 2, pVDAPEXΔC+pcDNA3; lane 3, pVDAPEXΔC+pcDNA3:furin; lane 4, pVDAPEXΔC+pcDNA3:PC5; lane 5, pVDAPEXΔC+pcDNA3:PC7. In the absence of the PC expression constructs, a single ˜33 kDa protein band was detected, corresponding to intact VEGF-DΔC (lane 2). Co-transfection with pVDAPEXΔC and pcDNA3:furin resulted in a single ˜21 kDa species representing the mature VHD (lane 3). Processing to generate the mature VHD was also detected in the conditioned medium of cells co-transfected with pVDAPEXΔC and the pcDNA3:PC5 (lane 4). However, co-transfection of pVDAPEXΔC and the pcDNA3:PC7 expression construct did not result in processing of VEGF-DΔC (lane 5).

These results demonstrate that furin and PC5 can promote cleavage of both the N-terminal and C-terminal propeptides from the VHD of VEGF-D whereas PC7 can only promote cleavage of the C-terminal propeptide.

### Example 5

The Mature Form of VEGF-D Generated By Furin Binds VEGFR-2 and VEGFR-3

The mature form of VEGF-D generated by 293EBNA cells is an activating ligand for the receptor tyrosine kinases VEGFR-2 and VEGFR-3 (Achen et al, Proc. Natl. Acad. Sci. USA 95 548-553, 1998). Therefore, receptor binding studies were carried out to establish if the mature form of VEGF-D generated by furin in transfected LoVo cells is a ligand for these receptors.

Binding to receptor extracellular domains was examined using soluble fusion proteins consisting of the extracellular domains of human VEGFR-2 or VEGFR-3 joined to the Fc portions of human IgG (referred to as VEGFR-2-Ig and VEGFR-3-Ig) (Achen et al, Proc. Natl. Acad. Sci. USA 95 548-553, 1998). Protein-A sepharose beads were incubated overnight with the conditioned media of 293EBNA cells expressing VEGFR-2-Ig or VEGFR-3-Ig to precipitate the soluble receptor constructs. The soluble receptors bound to protein-A sepharose beads were recovered by centrifugation (5 minutes, 250×g, 4° C.) and washed twice with binding buffer (0.5% (w/v) BSA, 0.02% (v/v) Tween 20, 10 μg/ml heparin in phosphate buffered saline). The soluble receptors bound to protein-A sepharose beads were then incubated with the conditioned media of LoVo cells, that had been co-transfected with pVDAPEX FULL-N-FLAG and pcDNA3:furin, for three hours at room temperature, and washed twice with binding buffer. Proteins bound to the VEGFR-2-Ig and VEGFR-3-Ig constructs were analysed by reducing SDS-PAGE and Western blotting with a biotinylated antibody against the VHD of human VEGF-D (R&D Systems), followed by Streptavidin-horse radish peroxidase conjugate (Zymed).

FIG. 6 shows the results of the receptor binding experiments using (A) VEGFR-2-Ig and (B) VEGFR-3-Ig. No mature VEGF-D species were detected in the medium of cells transfected with pAPEX3 and pcDNA3 (lane 1), nor with pVDAPEX FULL-N-FLAG and pcDNA3 (lane 2). Both VEGFR-2-Ig and VEGFR-3-Ig precipitated an ˜21 kDa VEGF-D species from the medium of LoVo cells transfected with pVDAPEX FULL-N-FLAG and pcDNA3:furin (lane 3; indicated by arrows). Sizes of molecular weight standards are shown to the left of each panel and asterisks indicate a non-specific band observed in all samples.

These results demonstrate that the mature form of VEGF-D generated by furin binds to the extracellular domains of VEGFR-2 and VEGFR-3, as does the previously characterised mature form generated by 293EBNA cells (Stacker et al, J. Biol. Chem. 274 32127-32136, 1999).

### Conclusions

This study demonstrates that PCs can promote activation of VEGF-D and identifies three members of this family of proteases capable of this process. Furin and PC5 promote cleavage of both propeptides of VEGF-D from the VHD, indicating that these enzymes can fully activate this growth factor. In contrast, PC7 promotes cleavage of the C-terminal propeptide, not the N-terminal propeptide, from the VHD. Hence, PC7 can only partially activate VEGF-D. The findings that PCs can be expressed in cancer (Khatib et al, Am. J. Pathol. 160 1921-1935, 2002), as can VEGF-C and VEGF-D, indicate that this proteolytic activation mechanism, that dramatically enhances the affinities of VEGF-C and VEGF-D for VEGFR-2 and VEGFR-3, may be important for promoting tumor angiogenesis and lymphangiogenesis, thereby facilitating solid tumor growth and metastatic spread. This proteolytic activation of these growth factors is therefore a promising target for novel anti-cancer therapeutics. Furthermore, the findings that PCs also activate other growth factors that can be important for tumor progression, such as TGF-β and PDGF-A (Dubois et al, J. Biol. Chem. 270 10618-10624, 1995; Siegfried et al, Cancer Res. 63 1458-1463, 2003), make the PCs even more attractive therapeutic targets.

For the sake of completeness of disclosure, all literature articles cited herein are expressly incorporated in this specification by reference.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of inhibiting angiogenesis or lymphangiogenesis comprising administering to an organism in need thereof an effective angiogenesis or lymphangiogenesis inhibiting amount of a proprotein convertase inhibitor.

2. A method according to claim 1, wherein said proprotein convertase inhibitor is selected from the group consisting of inhibitory prosegments of proprotein convertases, inhibitory variants of antitrypsin and peptidyl haloalkyl ketone inhibitors.

3. A method according to claim 2, wherein said proprotein convertase inhibitor comprises an inhibitory prosegement of a proprotein convertase selected from the group consisting of PACE4, PC1, PC2, PC3, PC4, PC5A, PC5B, PC6A, PC6B, PC7 and Furin

4. A method according to claim 3, wherein said proprotein convertase inhibitor comprises an inhibitory prosegment of Furin or PC7.

5. A method according to claim 2, wherein said proprotein convertase inhibitor is a peptidyl haloalkyl ketone inhibitor selected from the group consisting of decanoyl-Arg-Val-Lys-Arg-chloromethylketone (Dec-RVKR-CMK), decanoyl-Phe-Ala-Lys-Arg-chloromethylketone (Dec-FAKR-CMK), decanoyl-Arg-Glu-Ile-Arg-chloromethylketone (Dec-REIR-CMK), and decanoyl-Arg-Glu-Lys-Arg-chloromethylketone (Dec-REKR-CMK).

6. A method according to claim 5, wherein said proprotein convertase inhibitor comprises decanoyl-Arg-Val-Lys-Arg-chloromethylketone (Dec-RVKR-CMK).

7. A method according to claim 2, wherein said proprotein convertase inhibitor is an inhibitory variant of antitrypsin.

8. A method according to claim 7, wherein said proprotein convertase inhibitor comprises α-1 antitrypsin Portland.

9. A method according to claim 1, wherein said organism is suffering from a tumor.

10. A method according to claim 1, wherein said organism is suffering from macular degeneration.

11. A method according to claim 1, wherein said organism is a mammal.

12. A method according to claim 11, wherein said mammal is human.

13. A method of inhibiting angiogenesis or lymphangiogenesis comprising administering to an organism in need thereof an effective angiogenesis or lymphangiogenesis inhibiting amount of a proprotein convertase antagonist selected from the group consisting of an anti-proprotein convertase antibody, an antisense nucleic acid molecule against a polynucleotide coding for a proprotein convertase, and an siRNA for inhibiting proprotein convertase expression.

14. A pharmaceutical composition for inhibiting angiogenesis or lymphangiogenesis in a patient in need thereof, the composition comprising an effective angiogenesis or lymphangiogenesis inhibiting amount of a proprotein convertase inhibitor, or an antagonist selected from the group consisting of an anti-proprotein convertase antibody, an antisense nucleic acid molecule against a polynucleotide coding for a proprotein convertase, and an siRNA for inhibiting proprotein convertase expression, and a pharmaceutically acceptable excipient.

15. A method of treating a condition selected from the group consisting of cancer, tumor growth, tumor metastasis, macular degeneration, inflammatory mediated diseases, rheumatoid arthritis, diabetic retinopathy and psoriasis in a patient, said method comprising administering to said patient an effective amount of a pharmaceutical composition of claim 14.

16. A method according to claim 15, wherein the method is for inhibiting tumor growth and the pharmaceutical composition comprises an effective tumor growth inhibiting amount of a proprotein convertase inhibitor.

17. A method according to claim 15, wherein the method is for inhibiting cancer metastasis and the pharmaceutical composition comprises an effective tumor growth inhibiting amount of a proprotein convertase inhibitor.

18. A method according to claim 15, wherein the method is for treating an inflammatory disease and the pharmaceutical composition comprises an effective tumor growth inhibiting amount of a proprotein convertase inhibitor.

19. A method according to claim 18, wherein said inflammatory disease is rheumatoid arthritis.

20. A method according to claim 15, wherein said proprotein convertase inhibitor is selected from the group consisting of inhibitory prosegments of proprotein convertases, inhibitory variants of antitrypsin and peptidyl haloalkyl ketone inhibitors.

21. A pharmaceutical composition according to claim 14, wherein said proprotein convertase inhibitor is selected from the group consisting of inhibitory prosegments of proprotein convertases, inhibitory variants of antitrypsin and peptidyl haloalkyl ketone inhibitors.

22. A pharmaceutical composition according to claim 21, wherein said inhibitory prosegement of a proprotein convertase selected from the group consisting of PACE4, PC1, PC2, PC3, PC4, PC5A, PC5B, PC6A, PC6B, PC7 and Furin

23. A method according to claim 22, wherein said proprotein convertase inhibitor comprises an inhibitory prosegment of Furin or PC7.
